(19) United States
(12) Patent Application Publication
(10) Pub. No.: US 2013/0084587 A1
(43) Pub. Date: Apr. 4, 2013

(54) Levels of cytokeratins in blood and body fluids as biomarkers for cancer screening, diagnosis and treatment monitoring

(71) Applicants: Peilin Zhang, Hurricane, WV (US); Sylvia Tina Zhang, Hurricane, WV (US); Xianglin Shi, Lexington, KY (US)

(72) Inventors: Peilin Zhang, Hurricane, WV (US); Sylvia Tina Zhang, Hurricane, WV (US); Xianglin Shi, Lexington, KY (US)

(21) Appl. No.: 13/251,228

(22) Filed: Oct. 1, 2011

(51) Int. Cl. G01N 33/574

(52) U.S. Cl. 435/7.94

(57) ABSTRACT

Cytokeratins are intermediate filaments in the epithelial cells. Human cancers are malignant counterparts of normal epithelia. Human cancer cells express various kinds of cytokeratins dependent upon the specific cancer cell type. Pathologists have been using cytokeratin immunostaining for classification of tumor origins and cancer types. We claim that the protein levels of cytokeratin 7 (CK7) in the blood and/or body fluids are proportional to the tumor burden, and these protein levels of CK7 can be used as biomarkers for cancer screening, diagnosis and treatment monitoring.

### CROSS REFERENCES TO RELATED APPLICATION

None

### STATEMENT REGARDING FEDERALLY SPONSORED RESEARCH OR DEVELOPMENT

The idea and this research work were not sponsored by Federal agencies.

### BACKGROUND OF INVENTION

Cytokeratin (keratin) is a large family of closely related intermediate filaments in the epithelial cells serving diverse cellular functions. There are approximately 50 cytokeratin genes in human (1). A majority of the cytokeratins is found in skin and skin appendages, such as hair follicles. A number of cytokeratins are only present in the visceral epithelial cells and mesothelial cells. Cytokeratin 5, 6, 7, 8, 9, 18, 19 and 20 are predominantly expressed in mesothelial cells, lung epithelial cells, gastrointestinal tract, mammary glands, thyroid, urothelial cells and reproductive tract epithelia (2, 3). Extensive studies have been done by immunohistochemical staining on a variety of human carcinomas for the presence of various cytokeratins (2, 3). The current utility of cytokeratin immunostaining is primarily on the classification of cancer types and determination of cancer origin by anatomic pathologists (4). However, no study has been published as we know to date to assess the utility of protein levels of cytokeratin 7 in blood circulation and various body fluids for cancer screening, diagnosis and treatment monitoring. We at CHERSCO LLC tested to see if the blood levels of cytokeratin 7 are elevated in the cancer patients, and if these levels are reflective of the cancer, and can be used as a biomarker for cancer diagnosis and treatment monitoring. We have demonstrated in a small study that the protein levels of CK7 are elevated in the blood of lung cancer and breast cancer patients, and these CK7 protein levels are potential markers of lung cancer diagnosis and treatment monitoring. We are in the process of developing a system to measure the levels of the CK7 protein in the blood for potential marketing to the clinical hospital laboratories.

### BRIEF SUMMARY OF INVENTION

We have performed the preliminary “proof of concept” study to measure the level of the whole protein of CK7 in the blood of various cancer patients, and our data suggest that the level of CK7 protein in the blood and/or body fluids can potentially be used for diagnosis and treatment monitoring for lung cancer and breast cancer.

### DETAILED DESCRIPTION OF INVENTION

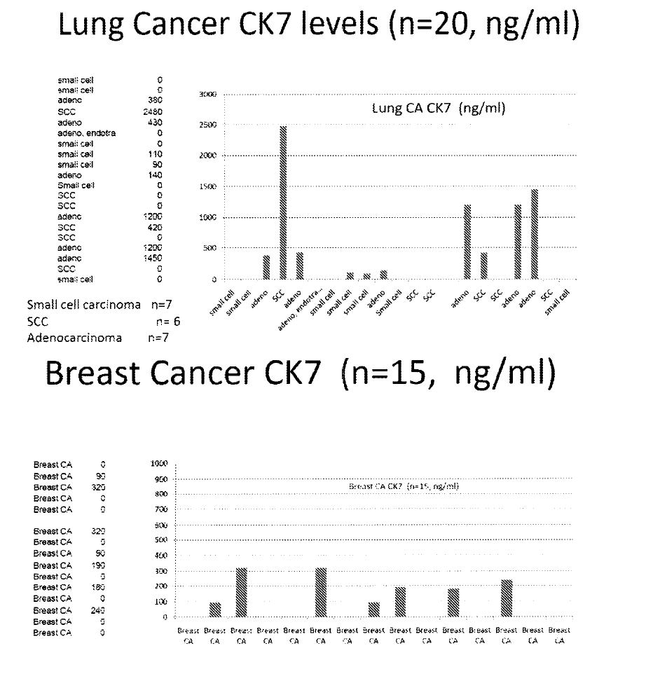

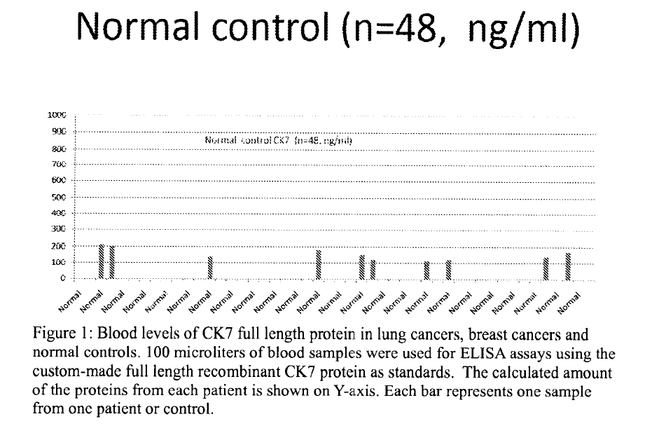

We have performed a small proof-of-concept study to measure the levels of CK7 in blood to see if the CK7 levels correlate with the patients' diagnoses of cancer. We have measured CK7 levels by Sandwich Enzyme-linked immunosorbent assay (ELISA assays) in patients with clear diagnoses of lung cancer, breast cancer, gastric cancer, and colon cancer using published methods. We have also included 48 normal controls. ELISA assay was performed as described extensively elsewhere. Briefly, a pair of antibodies against CK7 was purchased from Santa Cruz Biotechnologies, Inc. (Santa Cruz, Calif., Cat. # sc-25721, sc-70935). One antibody was used as a capturing antibody to coat the 96-well ELISA plate (Nunc. Cat. # 44-2404-21) and the other as a detecting antibody. The 96-well ELISA plate was blocked by 5% bovine serum albumin in phosphate-buffered saline (pH 7.4) for 1 hour. A sample of 100 microliter of plasma/serum was incubated with the capturing antibody at 4 degrees over night. The detecting method was as described using TMB substrate (Thermo Scientific, Cat. # MG157662). CK7 levels were calculated using standard curve and the standard full-length recombinant CK7 protein custom-made by Enzymax LLC (Enzymax, LLC, Lexington, Ky.). Our data indicate that the CK7 levels in the blood were significantly increased in lung cancer and breast cancer patients, but not in the normal controls (see FIG. 1). Furthermore, after surgery, the blood level of CK7 in two patient was dramatically decreased to normal level in 7 and 10 days (2 patients) (data not shown). These preliminary data indicate that the levels of CK7 in blood or possibly other body fluids, such as pleural fluid, ascites, vitreous fluid, and spinal fluids, can be used as biomarkers for diagnosis and treatment monitoring for lung cancer and breast cancer. In lung cancer patients, the CK7 levels are elevated only in the adenocarcinoma patients, and some fractions of small cell carcinoma and squamous cell carcinoma patients, consistent with our current clinical anatomic pathology practice and the previous immunohistochemical staining studies (2,3). In breast cancer patients, only 47% (7 of the 15 patients) patients with breast cancer show elevated levels of CK7 in their blood. Additionally, the blood CK7 levels are significantly higher in the lung cancer patients than those of the breast cancer patients.

It should be noted that we only measure the whole full length CK7 protein. We do not know if there is a CK7 fragment in the circulation, and our methods of measurement do not cover the partial CK7 protein or fragment of CK7 protein.

Based on our current data, we conclude that the CK7 levels in the blood greater than 100 ng/ml (or any particular value from the instruments, dependent upon the sensitivity of the system) are likely indicative of the presence of lung cancer or breast cancer. The CK7 levels in the blood could be used as a potential marker for lung cancer and breast cancer diagnosis or treatment monitoring. The level of CK7 protein in the blood as a diagnostic marker may vary dependent upon the sensitivity of the detection method. The potential use of CK7 levels in clinical setting is three folds: First, if any patient is clinically at high risk for lung cancer or breast cancer, elevated CK7 levels can assist diagnosis of cancer. CK7 cannot and will not replace the standard care of tissue diagnosis, but an elevated CK level in the blood in a high risk patient support the diagnosis of lung cancer or breast cancer. Second, if the patient has been clinically diagnosed with lung cancer or breast cancer by standard care method, and the patient is curretly under treatment, a simple blood test for CK7 levels may be used to monitor the treatment effect. If the CK7 levels are The current standard care of treamtent monitoring is by imaging study. Third, the CK7 levels in the blood vary from low (100 nanogram/ml range) to extremely high (microgram/ml range), and the high CK7 levels may serve as a prognosis marker to indicate poor prognosis of the cancer patient. We currently do not possess the data to support the claim for CK7 as prognosis marker. Further study will be needed in this regard. We do not have the data to support CK7 as marker for cancer screening. Further study will be needed as well.

We claim a method to use the CK7 levels in the blood and body fluid for diagnosis and treatment monitoring of lung cancer and breast cancer. Our method is immune based antibody-antigen reaction, and we only measure the whole protein levels of CK7, not any biologically degraded or processed CK7 fragments of any kind.

### REFERENCES CITED

1. Moll, R, Divo, M, and Langbcin, L The human keratins: biology and pathology. Histochem Cell Biol, 2008; 129 (6): 705-733.
2. Chu P., W, E., Weiss, L M. Cytokeratin 7 and cytokeratin 20 expression in epithelial neoiplasm: a survey of 435 cases. Modern Pathology, 2000; 13 (962-972.
3. Wang, N, et al Coordinated expression of cytokeratin 7 and 20 defines unique subsets of carcinomas. Applied Immunohistochemistry, 1995; 3 (99-107.
4. Rosai, J Rosai and Ackerman's Surgical Pathology, 10th edition: Mosby; 2011.

1. Method to detect the protein levels of cytokeratin 7 (CK7) in blood and/or body fluids by immune reaction as a biomarker for diagnosis and treatment monitoring of lung cancer and breast cancer.
